(19) United States
(12) Patent Application Publication
(10) Pub. No.: US 2005/0222403 A1
(43) Pub. Date: Oct. 6, 2005

(54) Oligonucleotide probes for in vitro, in vivo and intra-cellular detection

(76) Inventor: Frank E. Lyles, San Francisco, CA (US)

Correspondence Address: David M McConoughey;Stoll Miskin & Badie, Suite 4710, 350 Fifth Avenue, New York, NY, 10118-4710, US

(21) Appl. No.: 10/514,160

(22) PCT Filed: May 13, 2003
(86) PCT No.: PCT/US03/15362

§ 371 Date: Nov. 12, 2004

Related U.S. Application Data
(60) Provisional application No. 60/378,006, filed on May 13, 2002.

(51) Int. Cl. C12Q 1/68; C07H 21/04

(52) U.S. Cl. 536/24.3; 435/6

(57) ABSTRACT

A oligonucleotide probe containing specific probe and switch sequences for in vitro, in vivo and intracellular detection and identification of target sequences and molecules. Methods of using such probes for the detection of target sequences and molecules in various cells and tissues as well as a variety of biological samples and fluids. Methods of using such probes in non-clinical areas, such as agriculture.

### CROSS-REFERENCE TO RELATED APPLICATION(S)

The benefit of U.S. Patent Application Ser. No. 60/378,006, filed May 13, 2002 is claimed.

### FIELD OF THE INVENTION

The present invention relates generally to the field of oligonucleotide probes, especially as these are employed in bioassays relying on nucleic acid hybridization probes for the detection of specific genes, polynucleotide segments and RNA molecules. It also relates to the field of clinically useful assays of tissue, blood, and urine samples, as well as other biological materials and fluids.

### BACKGROUND OF THE INVENTION

The use of nucleic acid hybridization probes in bioassays is well known. (See, for example, Gillespie et al, A Quantitative Assay for DNA-RNA Hybrids with DNA Immobilized on a Membrane, J. Mol. Biol. 12:829-842 (1965)). In general, such an assay involves separating the nucleic acid polymer chains in a sample, as by melting or other means of denaturation, fixing the separated DNA strands to a nitrocellulose membrane, and then introducing a probe sequence which is complementary to a unique sequence of the material to be identified (called the “target”) and then incubating the mixture to allow the probe segments to hybridize to complementary target segments (if target polynucleotides are present). Non-hybridized probes are removed by washing and then the amount of probe remaining is determined by one of a variety of available techniques, thereby providing a measurement of the amount of target material in the sample.

A more recently developed form of bioassay that uses nucleic acid hybridization probes involves a second probe, often called a “capture probe.” (Ranki et al., Sandwich Hybridization as a convenient Method for the Detection of Nucleic Acids in Crude Samples, Gene 21:77-85 (1983); Syvanen et al., Fast Quantification of Nucleic Acid Hybrids by Affinity-based Hybrid Collection, Nucleic Acids Res. 14:5037-5048 (1986)) A capture probe contains a radiolabeled nucleic acid sequence complementary to the target, preferably in a region near the radioactively labeled probe. The capture probe often contains a site that reacts with the surface of a solid support so that hybridization can be carried out in solution, where it occurs rapidly, and the hybrids can then be bound to a solid surface. One example of such a means is biotin. (See: Langer et al., Enzymatic Synthesis of Biotin-Labeled Polynucleotides: Novel Nucleic Acid Affinity Probes, Proc. Natl. Acad. Sci. USA 78:6633-6637 (1981)) Through biotin the capture probe can be bound to streptavidin covalently linked to solid beads.

For all such techniques, a common feature (and common problem) is the means used to detect the probe once it is bound and how to measure only bound probe while avoiding interference by other molecules or by surrounding structures within the probe and/or target sequence. Several approaches have been used for detecting the probe. One is to link a readily detectable reporter group to the probe (such as a radioactive isotope, which may replace an atom normally in the probe or may be part of an extrinsic chemical grouping that does not interfere with the specificity of hybridization). Examples of reporter groups are fluorescent organic molecules and ³²P-labeled phosphate groups. These detection techniques have a practical limit of sensitivity of about a million targets per sample (meaning that fewer than this number per sample will commonly result in a failure to detect and/or discriminate target versus non-target because the signal to noise ratio is not high enough, for example, the signal to background ratio for radiolabeled probes).

A second approach to probe detection and discrimination is to link a signal generating system to the probe. Examples are enzymes such as peroxidase. Probes are then incubated with a color-forming substrate. (Leary et al., Rapid and Sensitive Colorimetric Method for Visualizing Biotin-Labeled DNA Probes Hybridized to DNA or RNA Immobilized on Nitrocellulose: Bio-Blots, Proc. Natl. Acad. Sci. USA 80:4045-4049 (1983)). Such amplification reduces the minimum number of target molecules which can be detected. As a practical matter, however, nonspecific binding of probes has limited the improvement in sensitivity as compared to radioactive tagging to roughly an order of magnitude, i.e., to a minimum of roughly 100,000 target molecules.

Yet another approach is to make many copies of the target itself by in vivo methods. (See, for example, Hartley et al., Bioassay for Specific DNA Sequences Using a Non-Radioactive Probe, Gene 49:295-302 (1986)). This can also be done in vitro using a technique called the “polymerase chain reaction” (PCR). This technique was reported in Saiki et al., Enzymatic Amplification of Beta-globin Genomic Sequences and Restriction Site Analysis for Diagnosis of Sickle Cell Anemia, Science 230:1350-1354 (1985); Saiki et al., Primer-directed Enzymatic Amplification of DNA With a Thermostable DNA Polymerase, Science 239:487-491 (1988); Erlich et al., Specific DNA Amplification, Nature 331:461-462 (1988), and Mullis et al., European Patent Application Publication Nos. 200362 and 201184 (see also U.S. Pat. Nos. 4,683,195 and 4,683,202). In PCR, the probe is complementary only to the beginning of a target sequence but, through an enzymatic process, serves as a primer for replication of an entire target. Each repetition of the process results in another doubling of the number of target sequences until a large number (perhaps a million copies or more) of identical targets are generated. At that point detectable probes, e.g., radioactively labeled probes, can be used to detect the amplified number of targets. The sensitivity of this method of target amplification is generally limited by the number of “false positive signals” generated, that is, generated segments that are not true copies of the target. Nonetheless, this method is quite sensitive. The procedure requires at least two nucleic acid probes and has three steps for a single cycle. This procedure is cumbersome and not always reliable.

Yet another method for amplification is to link to the probe an RNA that is known to be copied in an exponential fashion by an RNA-directed RNA polymerase. An example of such a polymerase is bacteriophage Q-beta replicase. (See: Haruna et al., Autocatalytic Synthesis of a Viral RNA In Vitro, Science 150:884-886 (1965)). Another example is brome mosaic virus replicase. (See: March et al., POSITIVE STRAND RNA VIRUSES, Alan R. Liss, New York (1987). In this technique, the RNA serves as a template for the exponential synthesis of RNA copies by a homologous RNA-directed RNA polymerase. The amount of RNA synthesized is much greater than the amount present initially. (See: Chu et al., Synthesis of an Amplifiable Reporter RNA for Bioassays, Nucleic Acids Res. 14:5591-5603 (1986); Lizardi et al., Exponential Amplification of Recombinant-RNA Hybridization Probes, Bio/Technology 6:1197-1203 (1988)). Replication of RNA, as opposed to target amplification using PCR, can be done in a single step. In that step one can synthesize as many as a billion copies of the replicatable RNA that was joined to the probe in as little as twenty minutes, which theoretically could lead to detection of a single target molecule. However, in practice the sensitivity of this type of probe replication is limited by the persistence of nonspecifically bound probes. Nonspecifically bound probes will lead to replication just as will probes hybridized to targets.

A major problem in this field has been the background signal produced by nonspecifically bound probe molecules. These background signals introduce an artificial limit on the sensitivity of bioassays. In conventional bioassays this problem is sometimes alleviated by the utilization of elaborate washing schemes that are designed to remove nonspecifically bound probes. These washing schemes have the disadvantage of adding to the complexity and cost of the assay while also presenting an absolute usage barrier in the case of living cells or in vivo analysis.

More recently, the “molecular beacon” has been devised in an attempt to overcome several of these obstacles. This structure uses a simple molecular allosteric switch that renders a nucleic acid hybridization probe capable, in an appropriate assay, of generating a signal only if the probe is hybridized to a target sequence, thereby greatly reducing sources of non-specific signal. Molecular beacons rely on the process of modulation of proximity dependent energy transfer processes, which may include fluorescent resonance energy transfer (FRET). In its most straightforward embodiment for use in hybridization studies, the beacon comprises a hairpin oligonucleotide having a fluorophore and a quencher molecule at its 5′- and 3′-ends (or in some other close proximity) so that the fluorophore is quenched and fluorescence is not detected. Quenchers commonly dissipate emission from the fluorophore by mechanical means without emitting any photons. When the probe hybridizes to the target (such as through its original hairpin loop region), the fluorophore and quencher are physically separated and the fluorophore is able to emit photons in response to excitation. The latter does not technically rely on the FRET process but could if the right donor and quencher pair were utilized.

Alternatively, in a more advanced embodiment, the quencher is replaced by an acceptor, whose emission is altered or enhanced when it is positioned a finite distance from a second fluorophore, called the donor (such as the fluorophore of the previous embodiment). At a specified distance (called a Förster radius) the emission by the acceptor (through the FRET process) is enhanced and the donor channels energy to the acceptor for emission of a detectable signal. The task of the labeled probe is to maintain the donor and acceptor either next to each other or at a distance much greater than the Förster radius, thereby thwarting the FRET process from operating. The FRET process will be optimal at the Förster radius between the donor and acceptor but will fall off with the sixth power of distance increases (more slowly as the distance decreases to zero). However, when the probe binds to the target, the donor and acceptor are thence positioned at their optimal distance for fluorescence resonance energy transfer and the desired signal is detected. In general, for the occurrence of the FRET process it is a necessary but not sufficient condition that the emission spectrum of the donor overlap the absorption spectrum of the acceptor (which is not at all required for a donor-quencher process since the latter does not require spectral overlap for the donor and quencher but could involve FRET if overlap occurs). This relationship is described by the overlap integral (which defines the probability of transfer) first described by T. Förster (Disc. Faraday Soc. 27:7-17 (1959)). Because of the dependence on separation of the donor:acceptor pair, such probes are highly advantageous for the measurement of molecular distances. A common donor-acceptor pair finding use in the hybridization area is pyrene-perylene (due to its high quantum efficiency and relatively short Förster distance (about 11-32 Angstroms)).

In a further improvement, a so-called “harvester” species has been used. Here, a fluorophore is located within a probe some distance from a potential emitter fluorophore but adjacent to a quencher such that energy transfer from the harvester to the quencher occurs instead of to the emitter and no fluorescence signal is detected until a conformational change, such as hybridization of the probe to a target, results in physical separation of the harvester from the quencher, thereby initiating energy transfer from the harvester to the emitter with resultant emission of a detectable signal. (See, for example, Tyagi S, Marras S A E, and Kramer F R, Wavelength-shifting molecular beacons. Nat Biotechnol 18,1191-1196 (2000)).

While molecular beacons have been useful in biological samples, living cells and in vivo applications present an obstacle in the form of nonspecific activation or instability of the molecular switch (possibly due to non-specific effects such as interactions with nucleic-acid binding proteins (Tsourkas A and Bao G., Detecting mRNA transcripts using FRET-enhanced molecular beacons. BED-Vol. 50, 2001 Bioengineering Conference-ASME (2001)), lipids and other components present in a living cell or the in vivo biological milieu. Due to such_effects, the use of molecular beacons in living cells leads to excessively high backgrounds that are not appropriate for fine analysis (Sokol et al., Real time detection of DNA:RNA hybridization in living cells. Proc Natl Acad Sci USA 95, 11538-11543 (1998)); Matsuo T., In situ visualization of mRNA for basic fibroblast growth factor in living cells. Biochimica Biophysica Acta 1379, 178-184 (1998)). Due to the nonspecific nature of these effects the activation of each molecular switch is transient-lasting for a much shorter amount of time than a legitimate (i.e., target-specific) activation event.

The present invention solves such problems by providing a means to couple multiple target-specific signal generating events within a molecular construct so that it is necessary to simultaneously activate multiple switches per molecular construct in order to generate an end signal. This coupling of multiple target-specific signal generating events may be used to significantly decrease the amount of illegitimate or background signal. In accomplishing this goal, the present invention provides probes containing an allosteric switch that are linked to any of a number of different signal generating systems whose activity is dependent on the state of the switch thereby facilitating assays of improved sensitivity that utilize the above constructs, as well as kits for performing such assays.

### BRIEF SUMMARY OF THE INVENTION

The present invention relates to an oligonucleotide probe for detecting a target polynucleotide. The oligonucleotide probe of the present invention comprises
a. first and second targeting portions, each targeting portion comprising a sequence complementary to at least one segment of the target polynucleotide and
b. a proximity-modulated signal generating system wherein, in a hybrid of the first and second targeting portions with the target polynucleotide, the hybrid comprises a plurality of changed conformations in the probe that activate the proximity-modulated signal generating system.

The proximity-modulated signal generating system may comprise an energy transfer system. In turn, the proximity-modulated signal generating system may comprise
at least one emitter and at least one quencher or
at least one emitter and at least one quencher and at least one harvester.

Further, the present invention relates to an oligonucleotide probe-target polynucleotide hybrid. The oligonucleotide probe-target polynucleotide hybrid of the present invention comprises
a. a target polynucleotide and
b. an oligonucleotide probe comprising
   i. first and second targeting portions, each targeting portion comprising a sequence complementary to at least one segment of the target polynucleotide and
   ii. a proximity-modulated signal generating system wherein the first and second targeting portions are hybridized with the target polynucleotide and
      wherein the hybrid comprises a plurality of changed conformations in the probe that activates the proximity-modulated signal generating system.

The proximity-modulated signal generating system may comprise an energy transfer system. The proximity-modulated signal generating system may comprise
at least one emitter and at least one quencher or
at least one emitter and at least one quencher and at least one harvester.

Further, the present invention relates to a method of detecting a target polynucleotide. The method of the present invention comprises
a. contacting the target polynucleotide with an oligonucleotide probe, the oligonucleotide probe comprising
i. first and second targeting portions, each targeting portion comprising a sequence complementary to at least one segment of the target polynucleotide and
ii. a proximity-modulated signal generating system whose activation requires hybridization of the first and second targeting portions with the target polynucleotide,
b. subjecting the probe to incident energy, and
c. detecting a signal generated by the probe.

The proximity-modulated signal generating system may be activated by at least one conformational change in the probe induced by hybridization of the probe with the target molecule. Further, the proximity-modulated signal generating system may be activated by a plurality of conformational changes in the probe induced by hybridization of the probe with the target molecule. The contacting may be performed under conditions promoting hybridization of the target polynucleotide with the first and second targeting portions. The proximity-modulated signal generating system may comprise an energy transfer system. The proximity-modulated signal generating system may comprise
at least one emitter and at least one quencher or
at least one emitter and at least one quencher and at least one harvester.

In one aspect the present invention relates to a method for determining the presence of a target polynucleotide comprising contacting said target polynucleotide with an oligonucleotide probe wherein said probe comprises a first targeting portion and a second targeting portion, wherein said first targeting portion comprises a quencher and an emitter spatially arranged so as to achieve quenching of said emitter and wherein said second targeting portion comprises a member selected from the group consisting of a quencher, a quencher and an emitter, and a harvester and wherein when said member is a quencher and an emitter these are spatially arranged so as to quench said emitter, and wherein said target and said probe are contacted under conditions promoting hybridization of said probe to said target and wherein said hybridization induces at least two conformational changes in said probe oligonucleotide resulting in separation of said quencher, or quenchers, from said emitter, or emitters, so as to produce a detectable signal indicative of hybridization to said target thereby determining the presence of said target polynucleotide.

In another aspect, the present invention relates to an oligonucleotide probe for detecting the presence of a target polynucleotide comprising first and second targeting portions each comprising a sequence complementary to at least one segment of said target polynucleotide, a quencher and an emitter positioned so as to effect quenching of said emitter by said quencher, and a harvester, wherein when said probe is hybridized to said target polynucleotide, said emitter and said quencher are positioned so that said emitter is not quenched and said harvester and said emitter are positioned so that said harvester emits light when said not-quenched emitter is contacted with electromagnetic radiation.

In a preferred embodiment thereof, said first targeting portion comprises at least two internally complementary segments, for example, where said first targeting portion is a hairpin oligonucleotide, most preferably wherein the first and second targeting portions each comprise segments that do not hybridize to each other.

In another preferred embodiment, the oligonucleotide probe of the invention further comprises a linker, or linker portion, positioned between said first and second targeting portions, most preferably wherein said linker is also positioned between said harvester and said emitter, especially wherein said linker comprises a sequence of oligonucleotides and most especially wherein said sequence of oligonucleotides does not hybridize to either said first or said second targeting portion.

In an alternative embodiment, said linker or linker portion is a polymer.

In one example of such an embodiment, the present invention relates to an oligonucleotide probe comprising first and second targeting portions and a linker portion, wherein said first targeting portion comprises a quencher and an emitter in sufficiently close spatial proximity to afford quenching of said emitter by said quencher, wherein said second targeting portion comprises a harvester and wherein said harvester and said emitter are separated by said linker portion which linker portion is different from said first or second targeting portion and wherein said harvester is such that it emits light when it is within a selected distance of said emitter and said emitter has been contacted with electromagnetic radiation of an excitatory wavelength.

In preferred embodiments of such probe the first targeting portion comprises at least two internally complementary segments, or switch portions, and is preferably a hairpin oligonucleotide wherein said switch portions hybridize with each other to form the duplex base of said hairpin. Preferably, the first targeting portion does not hybridize to the second targeting portion.

In preferred embodiments, the linker portion is any type of polymeric structure, such as an oligonucleotide, especially wherein this linker portion does not hybridize to either said first or said second targeting portion. The linker portion may also be other than an oligonucleotide.

In preferred embodiments, the emitter and harvester can form a FRET pair, preferably wherein the emitter is a member selected from the group consisting of fluorescein, BODIPY FL, EDANS, IAEDANS, Alexa Fluor 488, Alexa Fluor 532, Alexa Fluor 546, Alexa Fluor 555, Alexa Fluor 568 and Alexa Fluor 594, Alexa Fluor 633, Alexa Fluor 647, Alexa Fluor 660, Alexa Fluor 680, Alexa Fluor 700 and Alexa Fluor 750, Alexa Fluor 350 Alexa Fluor 430, Oregon Green 488, and Oregon Green 514 the quencher is a member selected from the group consisting of DABCYL and QSY™-7, QSY™-9, QSY™-21, in addition to any of a number of BlackHole™ dyes, and the harvester is a fluor that absorbs energy from the emitter and is excited poorly or not at all by a wavelength of light that will excite the emitter. The choice of harvester therefore depends upon the emitter used. As an example, when fluorescein is the emitter, Texas Red, 6-carboxyrhodamine 6G and tetramethylrhodamine are useful harvesters. Other useful harvesters include, without limitation, tetramethylrhodamine, fluorescein, Alexa Fluor 488, Alexa Fluor 532, Alexa Fluor 546, Alexa Fluor 555, Alexa Fluor 568 and Alexa Fluor 594, Alexa Fluor 633, Alexa Fluor 647, Alexa Fluor 660, Alexa Fluor 680, Alexa Fluor 700 and Alexa Fluor 750, Alexa Fluor 350 Alexa Fluor 430, Oregon Green 488, and Oregon Green 514. (All fluorophores and quenchers in this document are available from Molecular Probes, Eugene, Oreg. See http://www.idtdna.com/program/techbulletins/Fluorescent_Dye_Labeled_Oligonucleotid es.asp)

Preferably, the linker is of a length of at least twice the Förster radius for the harvester and emitter (i.e., for the FRET pair).

In a further aspect, the present invention relates to an oligonucleotide probe comprising first and second targeting portions, wherein each of said first and second targeting portions comprises a quencher and said probe further comprises an emitter sufficiently close to at least one of said quenchers to afford quenching of said emitter, and wherein said emitter emits light when it is separated from said quenchers and said emitter has been contacted with electromagnetic radiation of an excitatory wavelength.

In a preferred embodiment of such oligonucleotide probe, each of said first and second targeting portions comprises at least two internally complementary segments, most preferably wherein each of said first and second targeting portion forms a hairpin oligonucleotide. Preferably, said first targeting portion does not hybridize to said second targeting portion.

In a further embodiment, said quenchers each may have the same chemical structure or may be different in structure so long as they succeed in quenching the corresponding emitter when these are spatially separated by the requisite distance for quenching to occur.

In each of the embodiments of the invention, the probes may contain a single emitter and/or harvester and/or quencher, or the embodiment may contain a plurality of emitters and/or harvesters and/or quenchers.

In a further aspect, the present invention relates to an oligonucleotide probe comprising first and second targeting portions and a linker portion, wherein said first targeting portion comprises a first quencher and a first emitter in sufficiently close spatial proximity to afford quenching of said first emitter by said first quencher, wherein said second targeting portion comprises a second quencher and a second emitter in sufficiently close spatial proximity to afford quenching of said second emitter by said second quencher and wherein said first and second emitters are separated by said linker portion which linker portion is different from said first or second targeting portion and wherein said first and second emitters form a FRET pair.

In preferred embodiments, said first and second targeting portions each comprises at least two internally complementary segments, most preferably where these portions are each hairpin oligonucleotides and the complementary portions of one such targeting portion are not complementary to the complementary portions of the other targeting portion. In one such embodiment, no part of said first targeting portion hybridizes to any part of said second targeting portion.

In preferred embodiments, the linker portion is an oligonucleotide, or contains a sequence of nucleotides, or is not an oligonucleotide but some other type of polymeric structure. Where said linker is an oligonucleotide, or contains a sequence of nucleotides, it is preferable that it does not hybridize to either said first or said second targeting portion.

In a preferred embodiment of the probes of the invention, said probes are attached to a solid support.

In a preferred embodiment of the oligonucleotide probe of the invention the linker is of a length of at least twice the Förster radius for the harvester and emitter.

In a preferred embodiment thereof, the probes specifically disclosed herein are most advantageous, including where such probes are attached to a solid support. In other preferred embodiments the method is carried out in vivo, most preferably wherein said method is carried out inside a living cell, or is carried out in an animal, especially wherein said animal is a human patient.

### DEFINITIONS

Unless expressly stated otherwise herein, the following terms have the indicated definitions.

“Activated state” when used with reference to an oligonucleotide probe of the present invention means the structural and functional state of the oligonuleotide probe with respect to its signal generating activity on excitation, e.g., by energetic stimulation.

“Activation radius” or “activation distance” means the range of distances between two fluorophores that allows for efficient Fluorescence Resonance Energy Transfer (FRET) from the emitter fluor to the harvester fluor.

“Adjacent” (when used in reference to a segment) means that structures or sequences that are next to each other

“Conformation” means a particular three dimensional structure of a molecule that is kinetically and structurally distinct. It is most commonly the lowest energy structure of several possible alternative structures. “Conformational structure” means the same thing as “Conformation.”

“Fluor” or “fluorophore” refers to a fluorescent molecule or structure that emits photons in response to excitation by light of a specific wavelength.

“Förster Radius,” also called Ro, is the distance between FRET pairs that causes 50% of the excited donors to transfer their energy to the acceptor fluorophore.

“Fluorescence Resonance Energy Transfer” (FRET) is a process that shifts energy from an electronically excited molecule (emitter or donor fluorophore) to a nearby molecule (harvester, acceptor or quencher), returning the donor molecule to its ground state without emission of light from the donor.

“FRET pair” refers to a pair of emitters, such as an emitter and harvester, capable of generating a detectable signal through fluorescence resonance energy transfer when they are separated by a specified distance, optimally their respective Förster radius. The terms “FRET pair” and FRET partners” have a similar meaning.

“Hairpin” refers to a shape that is characterized by the hydrogen-bonding of complementary nucleic acids located at or near each end of the same nucleic acid strand. It resembles a string with a short segment of the ends twisted around one another.

“Harvester” refers to a fluorophore located within a probe some distance from an emitter fluorophore but adjacent to a quencher and whereby energy transfer from the harvester to the quencher is faster than to the emitter so that no fluorescence signal is detected until a conformational change, such as hybridization of the probe to a target, results in physical separation of the harvester from the quencher, thereby permitting the harvester to emit energy of a specific wavelength when it is a specific distance from the emitter.

“Hybridization” (as distinguished from nonspecific binding) means the binding of two strands of nucleic acid by way of hydrogen-bonds between complementary nucleic acid components known as nucleotides (GACT, G bonds with C and A bonds with T, in the usual Watson-Crick pairing mechanism).

“In vivo” and “in vivo milieu” refer to inside of a living cell, whether part of a living organism or in isolation, or in an organism.

“Linker” and “linker region” refer to a set or chain of molecules that connect, such as by covalent bonds, two or more molecules.

“Molecular sensor” means a sensor which is comprised of a single molecule.

“Molecular structure” means a higher order arrangement of set of atoms. A primary structure refers to the linear arrangement while secondary structure refers to the arrangement in three dimensional space.

“Plug-n-play” refers to having the ability to be used immediately without significant preparation or special tools.

“Proximity energy transfer” when used with reference to an oligonuleotide probe of the present invention means that the relative spatial proximity of at least one of the active elements of the signal generating system with respect to at least one other active element of the signal generating system permits energy transfer from one active element to another active element.

“Proximity modulated” when used with reference to an oligonuleotide probe of the present invention means that the signal generating capability of a signal generating system comprising the oligonucleotide probe is modulated by the relative spatial proximity of at least one of the active elements of the signal generating system with respect to at least one other active element of the signal generating system.

“Quantum efficiency” or “Q value” means the ratio of the number of photons emitted to number of photons absorbed by a fluor.

“Quenching moiety” or “quencher” means a molecule that receives from a donor energy via FRET and releases that energy in the form of heat or other means of mechanical energy dissipation rather by emitting light.

### BRIEF DESCRIPTION OF THE DRAWINGS

FIG. 1 is a schematic representation of one embodiment of the invention. Panel A shows a potential target sequence and probe of the invention that comprises a hairpin oligonucleotide, or first targeting portion, and a linker structure separating this from a second targeting portion comprising quencher and emitter structures wherein the quencher initially quenches the emitter while the linker separates the emitter and harvester. Panel B shows the probe bound to the target. After binding to a target polynucleotide, here by nucleotide complementarity, or hybridization, utilizing standard Watson-Crick rules, the emitter and quencher are separated by a sufficient distance that quenching is substantially reduced, resulting in fluorescence resonance energy transfer between the harvester and emitter to produce a detectable fluorescent signal (called a “terminal signal”) indicating presence of the target sequence in a sample. Thus, hybridization of the target sequences contained in the hairpin structure stably separates the quencher from the emitter while hybridization of the second target sequence brings the emitter and the harvester within the optimal FRET distance (i.e., the activation radius). The quencher eliminates energy from the emitter as heat when the two are in contact while the harvester emits light of a specific wavelength when it is within a the activation distance from the emitter. The probe depicted here is especially useful for in vivo measurements since it has the advantage of unimolecularly overcoming the effect of RNA binding proteins that increase the background in living cells by unfolding the beacon.

FIG. 2 is a schematic representation of one embodiment of the invention. Panel A shows a potential target sequence. Panel B shows an oligonucleotide probe of the invention wherein both first and second targeting portions are hairpin oligonucleotides, each with a terminal quencher and linked by a structure bearing an emitter so that quenching occurs. Panel C shows the probe bound to the target. After contact with a target, such as a target polynucleotide in a sample, the targeting portions hybridize to said target so that the quenchers are removed from the emitter and a detectable fluorescent signal results, thereby indicating presence of the target in the sample. Here, use of dual quenchers serves to lower the background despite transient conformational changes in each of the target loops. The probe depicted here is especially useful for in vivo measurements since it has the advantage of unimolecularly overcoming the effect of RNA binding proteins that increase the background in living cells by unfolding the beacon.

FIG. 3 is a schematic representation of an embodiment of the invention. Panel A shows a potential target sequence. Panel B shows an oligonucleotide probe of the invention comprising two hairpin oligonucleotides, each representing a targeting portion, and each possessing its own quencher-emitter pair, said emitters being linked by a linker. Panel C shows the probe bound to the target. After contact with a target, the hairpins hybridize to said target in such a way that the quenchers are removed from their respective emitters and the emitters are positioned in such respect to each other that they form a FRET pair and a detectable signal occurs. The linker is of such length (here, the Förster radius for the FRET pair) as to facilitate the FRET process and is sufficiently rigid to maintain the pair in the optimum dipole orientation. Thus, hybridization of the target sequences contained in the hairpin structures stably separate the quenchers from the fluorophores (i.e., emitter and harvester) while the hybridization of the second target sequence brings the emitter and the harvester within the optimal FRET distance (i.e., the activation radius). The probe depicted here is especially useful for in vivo measurements since it has the advantage of unimolecularly overcoming the effect of RNA binding proteins that increase the background in living cells by unfolding the beacon.

FIG. 4 shows an alternative embodiment of the type of probe depicted in FIG. 3.

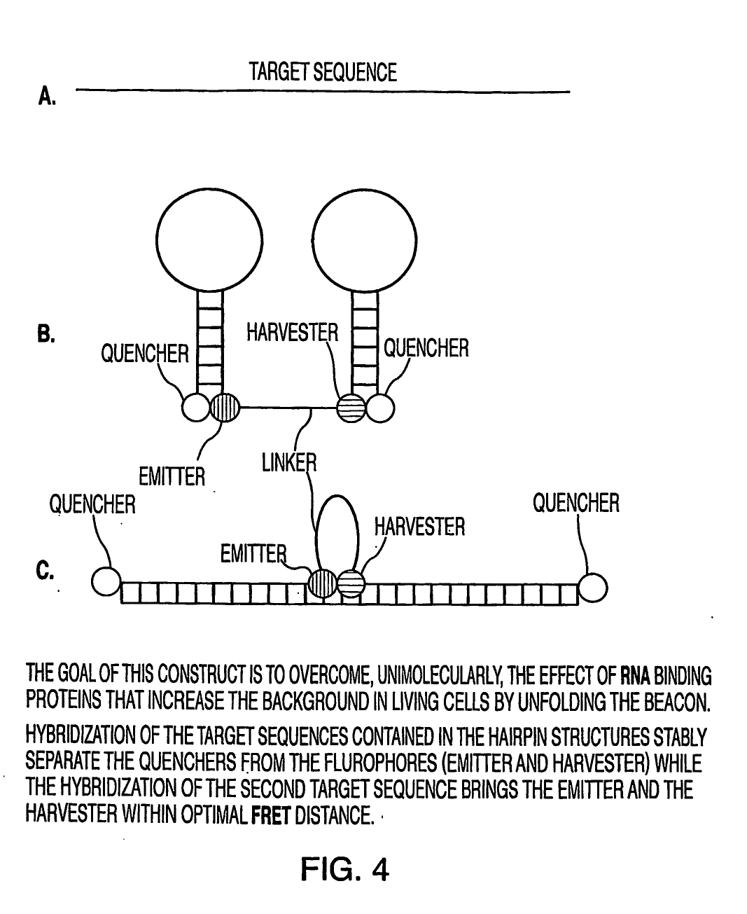

FIGS. 5A and 5B shows the structure of one example of an oligonucleotide probe of the invention.

### DETAILED DESCRIPTION OF THE INVENTION

In one aspect, the present invention relates to the present invention relates to an oligonucleotide probe for detecting the presence of a target polynucleotide comprising first and second targeting portions each comprising a sequence complementary to at least one segment of said target polynucleotide, a quencher and an emitter positioned so as to effect quenching of said emitter by said quencher, and a harvester, wherein when said probe is hybridized to said target polynucleotide, said emitter and said quencher are positioned so that said emitter is not quenched- and said harvester and said emitter are positioned so that said harvester emits light when said not-quenched emitter is contacted with electromagnetic radiation.

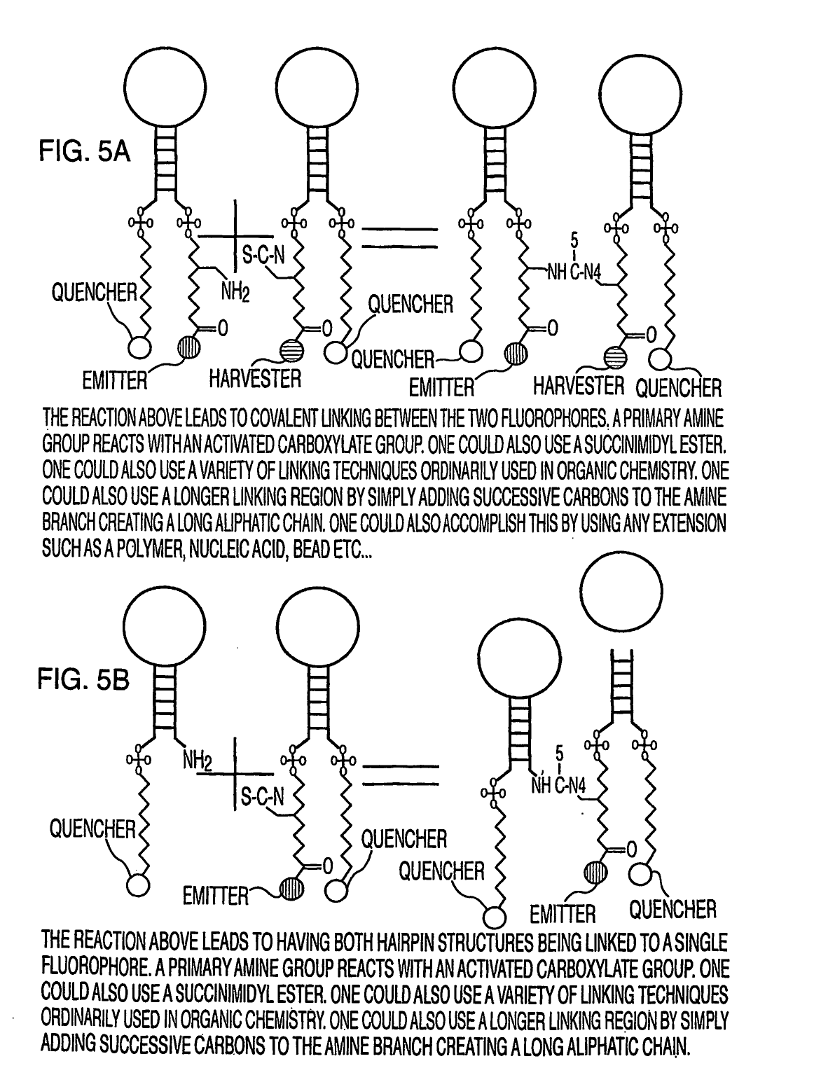

In a preferred embodiment thereof, said first targeting portion comprises at least two internally complementary segments, for example, where said first targeting portion is a hairpin oligonucleotide, most preferably wherein the first and second targeting portions each comprise segments that do not hybridize to each other. In an alternative embodiment, both targeting portions are hairpin oligonucleotides (see, for example, FIGS. 5 and 6).

In another preferred embodiment, the oligonucleotide probe of the invention further comprises a linker, or linker portion, positioned between said first and second targeting portions, most preferably wherein said linker is also positioned between said harvester and said emitter, especially wherein said linker comprises a sequence of oligonucleotides and most especially wherein said sequence of oligonucleotides does not hybridize to either said first or said second targeting portion.

In an alternative embodiment, said linker or linker portion is a polymer.

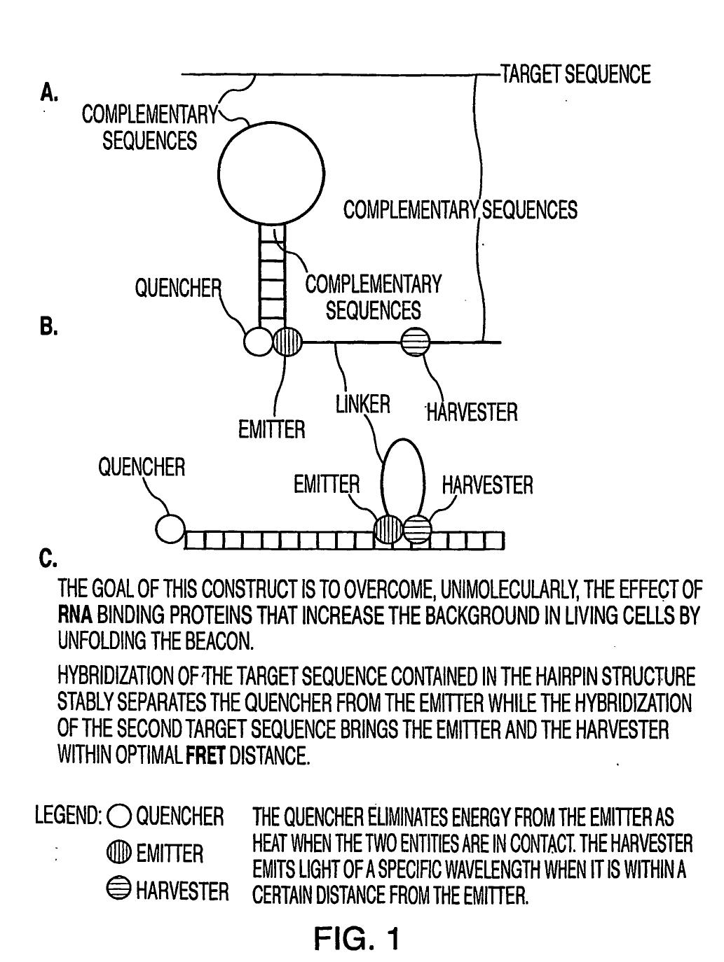

By way of non-limiting example, the present invention specifically contemplates the type of probe disclosed in FIG. 1, which represents an oligonucleotide probe comprising first and second targeting portions and a linker portion, wherein said first targeting portion comprises a quencher and an emitter in sufficiently close spatial proximity to afford quenching of said emitter by said quencher, wherein said second targeting portion comprises a harvester and wherein said harvester and said emitter are separated by said linker portion which linker portion is different from said first or second targeting portion and wherein said harvester is such that it emits light when it is within a selected distance of said emitter and said emitter has been contacted with electromagnetic radiation of an excitatory wavelength.

In preferred embodiments of such probe the first targeting portion comprises at least two internally complementary segments and is preferably a hairpin oligonucleotide and wherein said switch portions hybridize with each other to form the duplex base of said hairpin. Preferably, the first targeting portion does not hybridize to the second targeting portion.

In preferred embodiments, the linker portion is any type of polymeric structure, such as an oligonucleotide, especially wherein this linker portion does not hybridize to either said first or said second targeting portion. The linker portion may also be other than an oligonucleotide.

In preferred embodiments, the emitter and harvester can form a FRET pair, preferably wherein the emitter is a member selected from the group consisting of fluorescein, BODIPY FL, EDANS and IAEDANS, the quencher is a member selected from the group consisting of DABCYL and QSY™-7 in addition to any of a number of BlackHole™ dyes and the harvester depends upon the emitter used. For example, when fluorescein is the emitter, Texas Red, 6-carboxyrhodamine 6G and tetramethylrhodamine make useful harvesters.

Preferably, the linker is of a length of at least twice the Förster radius for the harvester and emitter (i.e., for the FRET pair).

In a further aspect, the present invention relates to an oligonucleotide probe comprising first and second targeting portions, wherein each of said first and second targeting portions comprises a quencher and said probe further comprises an emitter sufficiently close to at least one of said quenchers to afford quenching of said emitter, and wherein said emitter emits light when it is separated from said quenchers and said emitter has been contacted with electromagnetic radiation of an excitatory wavelength.

In a preferred embodiment of such oligonucleotide probe, each of said first and second targeting portions comprises at least two internally complementary segments, most preferably wherein each of said first and second targeting portion forms a hairpin oligonucleotide. Preferably, said first targeting portion does not hybridize to said second targeting portion.

In a further embodiment, said quenchers are the same chemical structure or may be different in structure so long as it succeeds in quenching the corresponding emitter when these are spatially separated by the requisite distance for quenching to occur.

In a further aspect, the present invention relates to an oligonucleotide probe comprising first and second targeting portions and a linker portion, wherein said first targeting portion comprises a first quencher and a first emitter in sufficiently close spatial proximity to afford quenching of said first emitter by said first quencher, wherein said second targeting portion comprises a second quencher and a second emitter in sufficiently close spatial proximity to afford quenching of said second emitter by said second quencher and wherein said first and second emitters are separated by said linker portion which linker portion is different from said first or second targeting portion and wherein said first and second emitters form a FRET pair.

In preferred embodiments, said first and second targeting portions each comprises at least two internally complementary segments, most preferably where these portions are each hairpin oligonucleotides and the complementary portions of one such targeting portion are not complementary to the complementary portions of the other targeting portion. In one such embodiment, no part of said first targeting portion hybridizes to any part of said second targeting portion.

In preferred embodiments, the linker portion is an oligonucleotide, or contains a sequence of nucleotides, or is not an oligonucleotide but some other type of polymeric structure. Where said linker is an oligonucleotide, or contains a sequence of nucleotides, it is preferable that it does not hybridize to either said first or said second targeting portion.

In a preferred embodiment of the oligonucleotide probe of the invention the linker is of a length of at least twice the Förster radius for the harvester and emitter.

The probes disclosed herein according to the invention are useful in applications both in vitro and in vivo, such as in cells or in animals, and may be used without attachment to any addition supporting structures. In addition, the probes of the invention are also highly useful when they are attached to a solid support.

In an additional aspect, the present invention relates to methods of using the probes of the invention for detecting a target polynucleotide.

In one embodiment thereof, the invention relates to a method for determining the presence of a target polynucleotide comprising contacting said target polynucleotide with an oligonucleotide probe wherein said probe comprises a first targeting portion and a second targeting portion, wherein said first targeting portion comprises a quencher and an emitter spatially arranged so as to achieve quenching of said emitter and wherein said second targeting portion comprises a member selected from the group consisting of a quencher, a quencher and an emitter, and a harvester and wherein when said member is a quencher and an emitter these are spatially arranged so as to quench said emitter, and wherein said target and said probe are contacted under conditions promoting hybridization of said probe to said target and wherein said hybridization induces at least two conformational changes in said probe oligonucleotide resulting in separation of said quencher, or quenchers, from said emitter, or emitters, so as to produce a detectable signal indicative of hybridization to said target thereby determining the presence of said target polynucleotide.

In a preferred embodiment thereof, the probes specifically disclosed herein are most advantageous, including where such probes are attached to a solid support. In other preferred embodiments the method is carried out in vivo, most preferably wherein said method is carried out inside a living cell, or is carried out in an animal, especially wherein said animal is a human patient.

In one embodiment thereof, the invention relates to a method for detecting a target polynucleotide comprising contacting said target polynucleotide with the oligonucleotide probe of claim 1, wherein each of said first and second targeting portions comprises a nucleotide sequence complementary to said target polynucleotide, under conditions promoting hybridization of said first and second targeting portions to said target polynucleotide, and wherein when said first and second targeting portions are hybridized to said target polynucleotide the emitter and quencher are separated by a sufficient distance to substantially reduce quenching and wherein said harvester and said emitter are separated by a sufficient distance to facilitate fluorescence resonance energy transfer (FRET) between said harvester and said emitter resulting in emission of electromagnetic radiation of a selected wavelength by said harvester and thereby determining the presence of said target polynucleotide.

In another embodiment, the invention relates to a method for detecting a target polynucleotide comprising contacting said target polynucleotide with the oligonucleotide probe of the invention wherein said first and second targeting portions each comprises a nucleotide sequence complementary to said target polynucleotide and under conditions promoting hybridization of said first and second targeting portions to said target polynucleotide, wherein when said first and second targeting portions are hybridized to said target polynucleotide the emitter and quenchers are separated by a sufficient distance such that quenching is substantially reduced and resulting in emission of electromagnetic radiation of a selected wavelength by said emitter and thereby determining the presence of said target polynucleotide.

In another embodiment, the present invention relates to a method for detecting a target polynucleotide comprising contacting said target polynucleotide with the oligonucleotide probe of the invention wherein said first and second targeting portions each comprises a nucleotide sequence complementary to said target polynucleotide and under conditions promoting hybridization of said first and second targeting portions to said target polynucleotide, wherein when said first and second targeting portions are hybridized to said target polynucleotide the first and second emitter are separated by a sufficient distance such that quenching is substantially reduced and wherein said first and second emitter are separated by a sufficient distance to facilitate fluorescence resonance energy transfer (FRET) between said first and second emitter resulting in emission of electromagnetic radiation of a selected wavelength and thereby determining the presence of said target polynucleotide.

The methods of the invention include preferred embodiments wherein said first targeting portion comprises a hairpin oligonucleotide having a loop portion and two switch portions wherein said switch portions are located at the ends of said hairpin oligonucleotide and said switch portions are complementary to each other and form a hybridized duplex sequence.

In a most preferred embodiment of such methods, the quencher is positioned at the 5′-end of said hairpin oligonucleotide and the emitter is positioned at the 3′-end of said hairpin oligonucleotide.

In other preferred embodiments of such methods, the loop portion of a hairpin oligonucleotide-shaped probe is complementary to a segment of said target polynucleotide, most preferably where the switch portions are not.

Commonly, said first targeting portion does not hybridize to said second targeting portion and, where a linker portion is present, it may or may not be an oligonucleotide. Where said linker is an oligonucleotide, or contains a sequence of nucleotides, it may hybridize to some portion of the target but more commonly it will not and is usually present only to spatially arrange emitters and/or harvesters and has no independent targeting ability. Conversely, the linker will not be an oligonucleotide and may be any desired polymeric structure that provides the requisite spatial separation.

In other specific embodiments of any of the methods of the invention, hybridization of the probe to the target results in the emitter and harvester being separated from each other by from 0.5 to 2 times the Förster radius for the harvester and emitter, preferably from 0.75 to 1.5 times the Förster radius for the harvester and emitter, most preferably from 0.9 to 1.25 times the Förster radius for the harvester and emitter, especially a distance of within 1 Angstrom of the Förster radius for the harvester and emitter, with the accepted Förster radius itself being the ideal value.

In preferred embodiments of the methods of the invention, such methods are carried out in vivo, most preferably inside of a cell or in an animal, especially a human, and wherein in the case of inside a cell said probes have been introduced into said cell by electroporation. For use in animals, the probes may be, and preferably are, attached to a solid support. Such solid support is a support found therapeutically acceptable for such uses and is made of pharmaceutically acceptable materials well known in the art.

The Invention allows for the analysis of gene expression in living cells, other in-vivo milieu or complex mixtures. The determination and quantification of RNA species in living cells requires a process that does not involve a rinsing step (that is usually required to wash away excess probe). Bearing this in mind, current methods to accomplish this feat involve the use of a reporter gene construct. However, such constructs can monitor only a few genes at one time and are limited to immortalized cell lines. Additionally, reporter-gene cell-lines are constructed and perpetuated with significant resources and time. They are not plug-n-play and as such require the researcher to engineer a new cell line for each cell-line that the researcher is interested in even if the same gene or set of genes is being studied.

Molecular beacons can overcome the above-mentioned drawbacks but they also have a major structural drawback. Molecular beacons are hairpin shaped nucleic acid probes that are designed to only emit a signal upon binding to their complementary partner Unfortunately, nucleic acid binding proteins also bind to molecular beacons and cause the hairpin-structure to open and allow the beacon to emit a signal in the absence of its intended target. This makes molecular beacons unsuitable for sensitive expression analysis.

The molecular sensors described herein overcome this problem by requiring two or more conformational changes to take place within the same molecule in order for the probe to emit a signal. The probability that multiple conformational changes will take place in the presence of a DNA binding protein or other non-target molecule absent target-specific binding is more remote than the single conformational change that takes place with a typical molecular beacon. This dependence of signal on at least two conformational changes, in turn, increases the signal to noise ratio and allows for greater signal resolution

For example, the process described in FIG. 1 requires at least two conformational changes to produce a signal. In one, hybridization of the first targeting portion to the target separates the emitter and quencher to facilitate fluorescence by the emitter. At the same time, the change produced by hybridization of the second targeting portion to the target brings the emitter and harvester within the activation distance for FRET to occur. A single conformation change, produced by non-specific protein binding inside a cell, would not result in this pattern of events and the wrong, if any, signal would be detected.

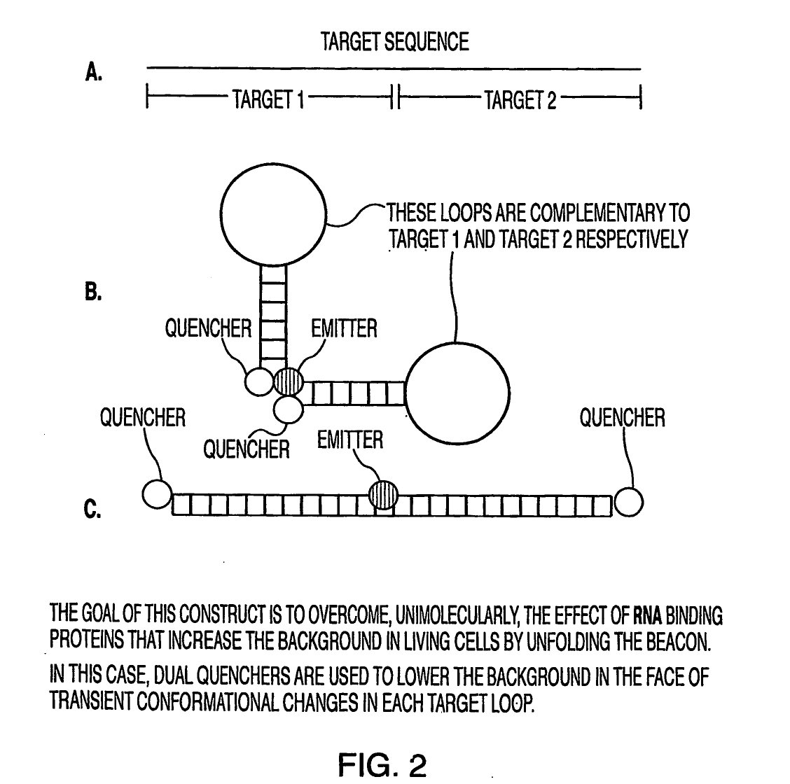

In the embodiment depicted in FIG. 2, two conformational changes are required: one to separate each of the quenchers from the emitter (or else emission does not occur).

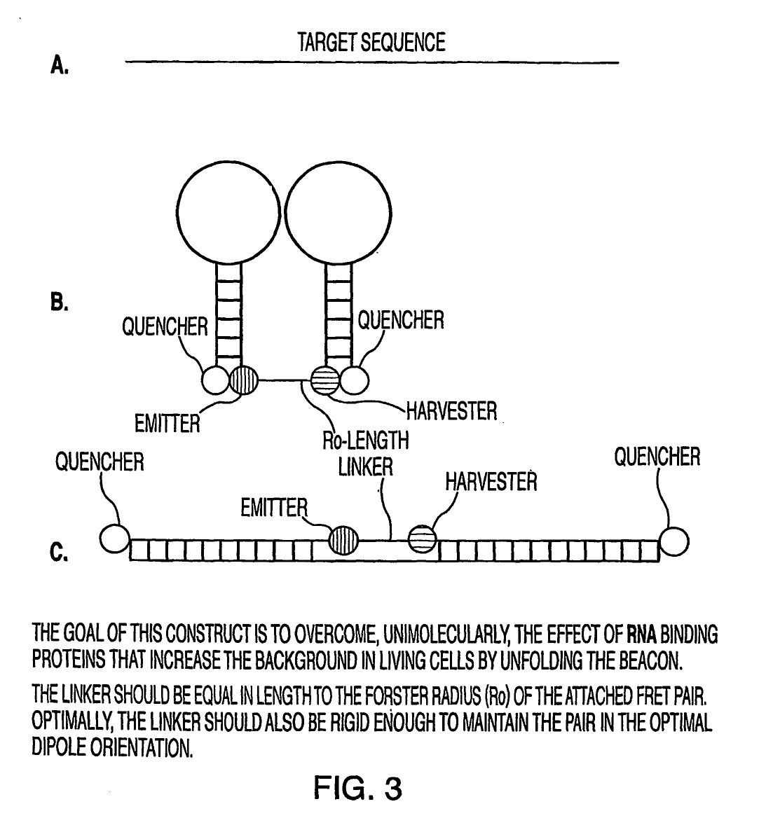

In the embodiment of FIG. 3, at least two conformational changes are required, each of which hybridizes a hairpin to the target and results in de-quenching of the respective emitter, thus providing the double emitter signal that indicates presence of the target. Where the emitters are different and each produces a different spectrum, non-specific unwinding due to protein binding will result in emission of only one of the emission spectra and not the composite of both that results from hybridization of both hairpins to the target polynucleotide.

The probes of the invention are also useful in developing molecular sensor designs wherein the conformational changes are unrelated or, or even opposite, in nature thereby greatly decreasing the probability that conformational changes will take place simultaneously under local molecular conditions.

Using the probes and methods of the invention, transcriptional level analysis can be done in living cells for drug discovery, pathway elucidation, or any other study in which it is important to isolate and recover individual cells that have tested positive. molecular beacons have excessive background noise for fine analysis but the probes of the invention reduce background to produce reliable data.

One application of the probes of the invention is drug discovery. By way of non-limiting example, where a gene, or genes, has (or have) been identified as being turned on by a specific drug, or class of drugs, various drug libraries are readily screened using one or more probes of the invention wherein said probe is complementary to the gene that is turned on by the drug. In the case of a DNA library, where a library of genes is being tested against a drug, one would first transfect the library into the appropriate cell line, thereby allowing each cell to make a single component of a library that is made up of millions of components. By electroporating the probe of the invention into the cell and then screening using, for example, a fluorescence-activated cell sorter (FACS) or other fluorescent analysis modality, one can identify the cells that have turned one or more genes on in response to the drug, thereby facilitating isolation and amplification of the affected genes.

As another non-limiting example of the applications of the probes and methods of the invention, it may be advantageous to identify a drug that activates the synthesis of a known protein, such as erythropoietin. Such determination is readily accomplished by screening microbead-based libraries that have been created by, for example, a split-pool or other type of synthetic method. Such libraries are typically produced on microbeads small enough to be electroporated into cells. As with a DNA library, target cells are isolated, beads extracted and the desired compound identified. For other applications, see Thelwell et al, Mode of action and application of Scorpion primers to mutation detection. Nucleic Acids Research, 28, 3752-3761 (2000); Steppan et al, The hormone resistin links obesity to diabetes. Nature 409, 307-312 (18 Jan. 2001).

One of the problems in using standard molecular beacons has been the presence of a high background in certain applications. This typically occurs in clinical samples that cannot be purged of the same factors (for example, nucleic acid binding proteins, lipids, and the like). The probes of the present invention solve this problem by reducing such background. In one non-limiting clinical diagnostic example, a probe of the invention is introduced into a patient where the probe is subject to non-target interactions that would otherwise cause previous probes to open and emit a false signal. The probe of the invention decreases such background signals thereby affording more accurate diagnosis while requiring lower doses of the diagnostic agent.

The invention can also be used in conjunction with a solid support and monitored internally. For example, a diagnostic target, such as RNA resulting from tissue or cellular breakdown, would be released from tumor cells in the intestine but is degraded before it reaches the feces making a cancer diagnosis difficult. In the case of gastrointestinal cancers, sloughed cells lyse and release contents which includes target RNA's that are predictive of malignancy. Typical molecular beacons would be affected by the same non-target interactions that produce a high background inside of a living cell. This background would make the device ineffective. The probes of the invention solve this problem. By covalently linking a large number of copies of the probes of the invention to a pill to be swallowed, the presence of the RNA is monitored via a fluorescent sensor contained within the pill. The sensor then transmits the signal to a device worn on the patient's body. The progress of a cancerous condition can likewise be monitored using such an arrangement.

In the same way, an intra-bladder device comprising the probes of the invention in solid form can detect recurrent bladder cancers.

In addition, such a device, present as a subcutaneous or intra-vascular device finds use in detecting infectious diseases that may be present by utilizing probes specific for targets in the genome of the disease organisms.

One embodiment of the present invention is depicted in FIG. 1, wherein the beacon comprises the three essential elements of such a probe, i.e., a probe sequence and complementary switch sequences on both sides of the probe and an additional switch sequence which uses an alternate switching mechanism.

In the case of fluorescent signaling, a “quencher” (black sphere in the figures) eliminates the signal from an “emitter” (light sphere) or a “harvester” (gray sphere) whenever it is in contact with one of these entities. An emitter emits “signal A” when stimulated with the proper wavelength of light (unless in contact with a quencher) and a harvester emits a “signal B” when it is in close proximity of “signal A”. Thus, where a construct contains an emitter, a harvester and quencher(s), “signal B” can only be generated if both the harvester and the emitter are in close proximity and no quencher is in contact with either the harvester or the emitter. In this case “signal B” is the detectable or “terminal signal” and one measures only “signal B” (see FIG. 1).

In an embodiment wherein there is just an emitter and quenchers but no harvester then one measures “signal A” and in order for “signal A” to be generated no quencher can be in contact with the emitter. In this case “signal A” is the “Terminal Signal” and the signal to be measured (see FIG. 2). The switch is “turned on” in the case that a “Terminal Signal” is generated and “turned off” in the case that a “Terminal Signal” is not generated.

In FIG. 1, the probe comprises a hairpin sequence having a 5′-end and a 3′-end. Immediately adjacent to the 5′-end of the probe sequence is a nucleic acid first switch sequence. Attached to the 3′ side of the probe sequence is a nucleic acid second switch sequence, here attached via a linker structure, which may or may not be an oligonucleotide. The first switch sequences are complementary and hybridize to each other via hydrogen bonds, forming the stem of a “hairpin” secondary structure. The second switch is made up of a sequence which binds to a sequence adjacent to the first target sequence. The emitter and harvester, forming a FRET pair, are joined by a linker that may include an oligonucleotide sequence, in which case the latter may be complementary to, or contain a portion that is complementary to, some part of the target such that, after the endpoint of hybridization of the probe to the target has been reached, the linker, whether hybridized to the target or not, serves to maintain the emitter and harvester within the activation radius for FRET to occur. The lower panel of FIG. 1 shows the probe sequence hybridized via hydrogen bonds to its predetermined target sequence (i.e., a target sequence of interest). First switch sequences are apart and not interacting with one another while the secondary switch sequence brings the signal elements within the activation radius (as depicted here, it is not hybridized to the target polynucleotide).

In a non-limiting application of the present invention, the probe molecules of the invention, including sequences complementary to a predetermined target sequence, are readily prepared by chemical synthesis using methods well known in the art, e.g., Gait, M. J., OLIGONUCLEOTIDE SYNTHESIS, IRL Press, Oxford, United Kingdom (1984). The probe contains a molecular beacon with an additional oligonucleotide (which is complementary to a segment of the target that is itself near the target sequence complementary to the loop region of the hairpin portion of the probe. This additional oligonucleotide is covalently attached to the “harvester” that is itself covalently positioned at the emitter through a oligomeric or polymeric structure, which may include an oligonucleotide or some type of long aliphatic side-chain. The probe of FIG. 1 may be synthesized using a primary amine group reacted with an activated carboxylate group both of which are contained in the side chains of the aliphatic chain of the moieties used to link the fluorophore to the nucleic acid. A succinimidyl ester synthesis is also available and known in the art. Further, a variety of linking techniques ordinarily used in organic chemistry are available and such methods in no way limit the breadth or utility of the invention. The length of the linker region is readily varied by adding successive carbons to the amine branch to create a long or short aliphatic chain. One example of a suitable chemical linkage is in FIG. 5.

Some available donors and acceptors (or quenchers) along with their Förster radii (in Angstroms) are as follows:

| Donor | Acceptor | Förster Radius |
|---|---|---|
| Fluorescein | Fluorescein | 44 |
| Fluorescein | QSY ™-7 | 61 |
| Fluorescein | Tetramethylrhodamine | 55 |
| EDANS | DABCYL | 33 |
| IAEDANS | Fluorescein | 46 |
| BODIPY FL | BODIPY FL | 57 |

Where the donor and acceptor are the same (such as with fluorescein) FRET can be detected as fluorescence depolarization. DABCYL, QSY™-7 and BlackHole™ dyes are “dark quenchers” and accept energy from the donor without emitting any photons.

Molecular sensors of the invention can be labeled with a variety of signal generating species, including, but not limited to, fluorophores, quenchers, enzymes, chemiluminescent molecules and the like. Fluorophores include, without limitation, acridine, AMCA, BODIPY, cascade blue, Cy2, edans, eosin, erythrosin, 6-Fam, fluorescein, rhodamine, tet, joe, hex, oregon green, tamra, rox, texas red, etc. and derivatives thereof. Quenchers include, but are not limited to, dabcyl, BHQ™ series, QSY™-series and derivatives thereof.

In another non-limiting application of the present invention, the probe contains two hairpin nucleic acid structures which are each attached to one fluor but they each have their own respective quencher. Both quenchers need to move away from the fluor in order for a signal to be generated. One method of synthesis is as follows:

Cytobeacon Synthesis (Illustrated in FIG. 5)

Oligonucleotides were synthesized from adenine, guanine, cytosine, and thymine phosphoramidates by a DNA automatic synthesizer (model 394, Perkin Elmer and a trityl hexylthiol linker (S-trityl-6-mercaptohexyl)-(2-cyanoethyl)-(N,N diisopropyl)-phosphoramidate) was linked to the 5′ end. Oligonucleotides were purified and fractionated with by high-pressure liquid chromatography and then dried and resuspended in phosphate buffered sodium (PBS). Target-1 oligos were then added to a 3× molar excess of DABCYL succinimidyl ester (Molecular Probes, Eugene, Oreg.) modified with an amino modifier (Amino Modifier C6 dT Phosphoramidite) at the 2-carbon branchpoint for 24 hours at room temperature. Excess DABCYL was removed via a Sephadex gel column and oligonucleotides were purified by HPLC. Target-2 oligos were modified to isothiocyanates, resuspended in PBS and added to Target-1 oligos in an isomolar mixture for 24 hours at room temperature

In an additional non-limiting application of the present invention, the probe contains two molecular beacons with FRET pair fluorophores separated by a short linker that allows the FRET pair to remain within the activation radius at all times. The method of synthesis is illustrated in FIG. 7.

In a further non-limiting application of the present invention, the probe is similar to the latter example but instead uses a longer linker region so as to only pull the emitter and harvester together when both loops are positioned at their respective targets.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The invention is described in more detail in the following non-limiting examples. It is to be understood that these methods and examples in no way limit the invention to the embodiments described herein and that other embodiments and uses will no doubt suggest themselves to those skilled in the art.

### EXAMPLE 1

### Transfection Detection

Described below is a method for detecting Phage Transfection in Mammalian cells. Only 1-3 percent of cells are expected to be successfully transfected via the following transfection procedure. For the procedure to be useful it is necessary to isolate that small percentage of cells without destroying their viability. Prior Methods require the use of phage that has been engineered with a Green Fluorescent Protein (GFP) reporter gene. The method below bypasses this non-trivial process by analyzing the expression of the transfected gene of interest (in this case epidermal growth factor (EGF) rather than relying on a reporter gene.

Materials and Methods

Phage Transfection

Cell Lines. K562 human leukemia cells were obtained from the American Type Culture Collection. DNA encoding EGF (epidermal growth factor) was inserted in to an M13 phage vector. Phage were purified and banded by PEG precipitation and CsCl ultracentrifugation. The phage were added to a suspension of K562 cells and incubated at 37% for 72 hours

Cytobeacon Synthesis

Oligonucleotides were synthesized from adenine, guanine, cytosine, and thymine phosphoramidates by a DNA automatic synthesizer (model 394, Perkin Elmer and a trityl hexylthiol linker (S-trityl-6-mercaptohexyl)-(2-cyanoethyl)-(N,N diisopropyl)-phosphoramidate) was linked to the 5′ end. Oligonucleotides were purified and fractionated with by high-pressure liquid chromatography and then dried and resuspended in phosphate buffered sodium (PBS). Target-1 oligos were then added to a 3× molar excess of DABCYL succinimidyl ester (All reactive fluorophores were purchased from Molecular Probes, Eugene, Oreg.) modified with an amino modifier (Amino Modifier C6 dT Phosphoramidite) at the 2-carbon branchpoint for 24 hours at room temperature. Excess DABCYL was removed via a Sephadex gel column and oligonucleotides were purified by HPLC. Texas Red and fluorescein were added to Target-2 oligos and Target-1 oligos, respectively, by reacting their acetamides with a 5′-thiol group and the resultant Target-2 oligos were modified to isothiocyanates, resuspended in PBS and added to the resultant Target-1 oligos in an isomolar mixture for 24 hours at room temperature. This Cytobeacon synthesis involves a direct linkage of the fluorophore sidechains and is detailed graphically in FIG. 7. Target-1 oligos consist of the sequence GCGTGGGCGATGGTT, Target-2 oligos consist of the sequence GTTGTCATTGTCGGC. These sequences are complementary to directly adjacent sequences of EGF-mRNA.

Electroporation and Analysis

After 72 hours of incubation with phage, K562 cells were centrifuged at 300 rpm for 10 min and re-suspended into RPMI 1640 medium with 5% bovine calf serum at 37 degrees Centigrade to a final concentration of 100,000 cells/ml. Cytobeacon probes were added to the cell suspension at a final concentration of 1 ng/ml and the suspension was kept in a 37 degrees Centigrade water bath for 10 minutes. The suspension was then subjected to rotary electroporation at 1.85 kv/cm. The cells were then loaded into a fluorescence activated cell sorter (MoFlo, Cytomation) with the primary laser tuned to excite at 488 nm. The emitter, fluorescein, emits at 520 nm upon excitation while the harvester, Texas Red is not excited by 488 nm. Texas Red, does however, emit at 609 nm when in it is within approximately 100 Angstroms of excited fluorescein. Upon analysis at 609 NM a first peak is expected at 3-5× background fluorescence (This peak corresponds to negative cells) a second peak is expected at 40-50× background fluorescence (This peak corresponds to positive cells). The first peak corresponds to cells which contain beacon but not the EGF-mRNA target. This peak is due to transient beacon activation. The percentage of cells found to be transfected with EFG bearing M13 (Cells isolated from the second peak) should be between 1-3%.

1. An oligonucleotide probe for detecting a target polynucleotide, said oligonucleotide probe comprising
a. first and second targeting portions, each targeting portion comprising a sequence complementary to at least one segment of said target polynucleotide and
b. a proximity-modulated signal generating system wherein, in a hybrid of said first and second targeting portions with said target polynucleotide, the hybrid comprises a plurality of changed conformations in said probe that activate said proximity-modulated signal generating system.

2. The oligonucleotide probe of claim 1 wherein said proximity-modulated signal generating system comprises an energy transfer system.

3. The oligonucleotide probe of claim 1 wherein said proximity-modulated signal generating system comprises
at least one emitter and at least one quencher or
at least one emitter and at least one quencher and at least one harvester.

4. An oligonucleotide probe-target polynucleotide hybrid comprising
a. a target polynucleotide and
b. an oligonucleotide probe comprising
i. first and second targeting portions, each targeting portion comprising a sequence complementary to at least one segment of said target polynucleotide and
ii. a proximity-modulated signal generating system
wherein said first and second targeting portions are hybridized with said target polynucleotide and
wherein said hybrid comprises a plurality of changed conformations in said probe that activate said proximity-modulated signal generating system.

5. The oligonucleotide probe-target polynucleotide hybrid of claim 4 wherein said proximity-modulated signal generating system comprises an energy transfer system.

6. The oligonucleotide probe-target polynucleotide hybrid of claim 4 wherein said proximity-modulated signal generating system comprises
at least one emitter and at least one quencher or
at least one emitter and at least one quencher and at least one harvester.

7. A method of determining the presence or amount of a target polynucleotide comprising
a. contacting said target polynucleotide with an oligonucleotide probe, said oligonucleotide probe comprising
i. first and second targeting portions, each targeting portion comprising a sequence complementary to at least one segment of said target polynucleotide and
ii. a proximity-modulated signal generating system whose activation requires hybridization of said first and second targeting portions with said target polynucleotide,
b. subjecting said probe to incident energy, and
c. detecting a signal generated by said probe.

8. The method of claim 7 wherein said proximity-modulated signal generating system is activated by at least one conformational change in said probe induced by hybridization of the probe with the target molecule.

9. The method of claim 7 wherein said proximity-modulated signal generating system is activated by a plurality of conformational changes in said probe induced by hybridization of the probe with the target molecule.

10. The method of claim 7 wherein said contacting is performed under conditions promoting hybridization of said target polynucleotide with said first and second targeting portions.

11. The oligonucleotide probe of claim 7 wherein said proximity-modulated signal generating system comprises an energy transfer system.

12. The oligonucleotide probe of claim 7 wherein said proximity-modulated signal generating system comprises
at least one emitter and at least one quencher or
at least one emitter and at least one quencher and at least one harvester.

13. An oligonucleotide probe for detecting the presence of a target polynucleotide comprising first and second targeting portions each comprising a sequence complementary to at least one segment of said target polynucleotide, a quencher and an emitter positioned so as to effect quenching of said emitter by said quencher, and a harvester, wherein when said probe is hybridized to said target polynucleotide, said emitter and said quencher are positioned so that said emitter is not quenched and said harvester and said emitter are positioned so that said harvester emits light when said not-quenched emitter is contacted with electromagnetic radiation.

14. The oligonucleotide probe of claim 13 wherein said first targeting portion comprises at least two internally complementary segments.

15. The oligonucleotide probe of claim 13 wherein said first targeting portion is a hairpin oligonucleotide.

16. The oligonucleotide probe of claim 13 wherein said first and second targeting portions each comprise segments that do not hybridize to each other.

17. The oligonucleotide probe of claim 13 further comprising a linker positioned between said first and second targeting portions.

18. The oligonucleotide probe of claim 13 wherein said linker is also positioned between said harvester and said emitter.

19. The oligonucleotide probe of claim 13 wherein said linker comprises a sequence of nucleotides that does not hybridize to either said first or said second targeting portion.

20. The oligonucleotide probe of claim 19 wherein said linker portion is a polymer.

21. The oligonucleotide probe of claim 13 wherein said emitter is a member selected from the group consisting of fluorescein, BODIPY FL, EDANS and IAEDANS

22. The oligonucleotide probe of claim 13 wherein said harvester is a member selected from the group consisting of tetramethylrhodamine, fluorescein, DABCYL, BODIPY FL, and QSY™-7.

23. The oligonucleotide probe of claim 13 wherein said quencher is a member selected from the group consisting of DABCYL, QSY™-7 and a BlackHole™ dye.

24. The oligonucleotide probe of claim 13 wherein said linker is of a length of at least half but not more than twice the Förster radius for the harvester and emitter.

25. The oligonucleotide probe of claims 13-24 wherein said probe is attached to a solid support.

26. An oligonucleotide probe comprising first and second targeting portions, wherein each of said first and second targeting portions comprises a quencher and said probe further comprises an emitter sufficiently close to at least one of said quenchers to afford quenching of said emitter, and wherein said emitter emits light when it is separated from said quenchers and said emitter has been contacted with electromagnetic radiation of an excitatory wavelength.

27. The oligonucleotide probe of claim 26 wherein each of said first and second targeting portions comprises at least two internally complementary segments.

28. The oligonucleotide probe of claim 27 wherein each of said first and second targeting portions is a hairpin oligonucleotide.

29. The oligonucleotide probe of claim 28 wherein said first targeting portion does not hybridize to said second targeting portion.

30. The oligonucleotide probe of claim 28 wherein each of said quenchers has the same chemical structure.

31. The oligonucleotide probe of claim 28 wherein said emitter is a member selected from the group consisting of fluorescein, BODIPY FL, EDANS and IAEDANS

32. The oligonucleotide probe of claim 27 wherein said quenchers are selected from the group consisting of DABCYL, QSY™-7 and a BlackHole™ dye.

33. The oligonucleotide probe of claims 26-32 wherein said probe is attached to a solid support.

34. An oligonucleotide probe comprising first and second targeting portions and a linker portion, wherein said first targeting portion comprises a first quencher and a first emitter in sufficiently close spatial proximity to afford quenching of said first emitter by said first quencher, wherein said second targeting portion comprises a second quencher and a second emitter in sufficiently close spatial proximity to afford quenching of said second emitter by said second quencher and wherein said first and second emitters are separated by said linker portion which linker portion is different from said first or second targeting portion and wherein said first and second emitters form a FRET pair.

35. The oligonucleotide probe of claim 34 wherein said first targeting portion comprises at least two internally complementary segments.

36. The oligonucleotide probe of claim 34 wherein said first targeting portion is a hairpin oligonucleotide.

37. The oligonucleotide probe of claim 34 wherein said second targeting portion comprises at least two internally complementary segments.

38. The oligonucleotide probe of claim 34 wherein said second targeting portion is a hairpin oligonucleotide.

39. The oligonucleotide probe of claim 34 wherein said first targeting portion does not hybridize to said second targeting portion.

40. The oligonucleotide probe of claim 34 wherein said linker portion contains a sequence of nucleotides.

41. The oligonucleotide probe of claim 34 wherein said linker portion does not hybridize to either said first or said second targeting portion.

42. The oligonucleotide probe of claim 34 wherein said linker portion is other than an oligonucleotide.

43. The oligonucleotide probe of claim 34 wherein said linker portion is a polymer.

44. The oligonucleotide probe of claim 34 wherein said emitters are selected from the group consisting of fluorescein, BODIPY FL, EDANS and IAEDANS.

45. The oligonucleotide probe of claim 34 wherein said quenchers are selected from the group consisting of DABCYL, QSY™-7 and a BlackHole™ dye.

46. The oligonucleotide probe of claim 34 wherein said linker is of a length of at least half but not more than twice the Förster radius for the harvester and emitter.

47. The oligonucleotide probe of claims 34-46 wherein said probe is attached to a solid support.

48. A method for detecting a target polynucleotide comprising contacting said target polynucleotide with the oligonucleotide probe of claim 13, wherein said first and second targeting portions comprises a nucleotide sequence complementary to said target polynucleotide, under conditions promoting hybridization of said first and second targeting portions to said target polynucleotide, wherein when said first and second targeting portions are hybridized to said target polynucleotide the emitter and quencher are separated by a sufficient distance to substantially reduce quenching and wherein said harvester and said emitter are separated by a sufficient distance to facilitate fluorescence resonance energy transfer (FRET) between said harvester and said emitter resulting in emission of electromagnetic radiation of a selected wavelength by said harvester and thereby determining the presence of said target polynucleotide.

49. The method of claim 48 wherein said first targeting portion comprises a hairpin oligonucleotide having a loop portion and two switch portions wherein said switch portions are located at the ends of said hairpin oligonucleotide and said switch portions are complementary to each other and form a hybridized duplex sequence.

50. The method of claim 49 wherein said quencher is positioned at the 5′-end of said hairpin oligonucleotide and said emitter is positioned at the 3′-end of said hairpin oligonucleotide.

51. The method of claim 49 wherein the loop portion of said hairpin oligonucleotide is complementary to a segment of said target polynucleotide.

52. The method of claim 50 wherein the switch portions of said hairpin oligonucleotide are not complementary to said target polynucleotide.

53. The method of claim 48 wherein said first targeting portion does not hybridize to said second targeting portion.

54. The method of claim 48 wherein said linker portion contains a sequence of nucleotides.

55. The method of claim 53 wherein said linker portion hybridizes to said target polynucleotide.

56. The method of claim 48 wherein hybridization of the probe to the target results in the emitter and harvester being separated from each other by from 0.1 to 2.0 times the Förster radius for the harvester and emitter.

57. The method of claim 48 wherein hybridization of the probe to the target results in the emitter and harvester being separated from each other by from 0.25 to 1.5 times the Förster radius for the harvester and emitter.

58. The method of claim 48 wherein hybridization of the probe to the target results in the emitter and harvester being separated from each other by from 0.75 to 1.25 times the Förster radius for the harvester and emitter.

59. The method of claim 48 wherein hybridization of the probe to the target results in the emitter and harvester being separated from each other by a distance of within 1 Angstrom of the Förster radius for the harvester and emitter.

60. The method of claims 48-59 wherein said method is carried out in vivo.

61. The method of claims 60 wherein said target polynucleotide is located inside a cell and said oligonucleotide probes have been introduced into said cell.

62. The method of claim 61 wherein said probes have been introduced into said cell by electroporation.

63. The method of claims 48-59 wherein said probes are attached to a solid support.

64. A method for detecting a target polynucleotide comprising contacting said target polynucleotide with the oligonucleotide probe of claim 13 wherein said first and second targeting portions each comprises a nucleotide sequence complementary to said target polynucleotide and under conditions promoting hybridization of said first and second targeting portions to said target polynucleotide, wherein when said first and second targeting portions are hybridized to said target polynucleotide the emitter and quenchers are separated by a sufficient distance such that quenching is substantially reduced and resulting in emission of electromagnetic radiation of a selected wavelength by said emitter and thereby determining the presence of said target polynucleotide.

65. The method of claim 64 wherein each of said first and second targeting portions comprises a separate hairpin oligonucleotide each comprising a loop portion and two switch portions and wherein said switch portions are located at the ends of said hairpin oligonucleotide and said switch portions are complementary to each other and form a hybridized duplex sequence.

66. The method of claim 65 wherein said loop portions hybridize to said target polynucleotide.

67. The method of claim 66 wherein said switch portions do not hybridize to said target polynucleotide.

68. The method of claim 64 wherein said quenchers are the same chemical structure.

69. The method of claim 64 wherein said quenchers are chemically different.

70. The method of claims 64-69 wherein said method is carried out in vivo.

71. The method of claims 70 wherein said target polynucleotide is located inside a cell and said oligonucleotide probes have been introduced into said cell.

72. The method of claim 71 wherein said probes have been introduced into said cell by electroporation.

73. The method of claims 64-69 wherein said probes are attached to a solid support.

74. A method for detecting a target polynucleotide comprising contacting said target polynucleotide with the oligonucleotide probe of claim 34 wherein said first and second targeting portions each comprises a nucleotide sequence complementary to said target polynucleotide and under conditions promoting hybridization of said first and second targeting portions to said target polynucleotide, wherein when said first and second targeting portions are hybridized to said target polynucleotide the first and second emitter are separated by a sufficient distance such that quenching is substantially reduced and wherein said first and second emitter are separated by a sufficient distance to facilitate fluorescence resonance energy transfer (FRET) between said first and second emitter resulting in emission of electromagnetic radiation of a selected wavelength and thereby determining the presence of said target polynucleotide.

75. The method of claim 74 wherein said first and second targeting portion each comprises a hairpin oligonucleotide having a loop portion and two switch portions and wherein said switch portions are located at the ends of said hairpin oligonucleotide and said switch portions are complementary to each other and form a hybridized duplex sequence.

76. The method of claim 75 wherein said quencher is positioned at the 5′-end of said hairpin oligonucleotide and said emitter is positioned at the 3′-end of said hairpin oligonucleotide.

77. The method of claim 75 wherein the loop portion of each of said hairpin oligonucleotides is complementary to said target polynucleotide.

78. The method of claim 75 wherein the switch portions of said hairpin oligonucleotides are not complementary to said target polynucleotide.

79. The method of claim 78 wherein said linker portion contains a sequence of nucleotides.

80. The method of claim 74 wherein said linker portion hybridizes to said target polynucleotide.

81. The method of claim 74 wherein hybridization of the probe to the target results in the emitters being separated from each other by from 0.5 to 2 times the Förster radius.

82. The method of claim 74 wherein hybridization of the probe to the target results in the emitters being separated from each other by from 0.75 to 1.5 times the Förster radius.

83. The method of claim 74 wherein hybridization of the probe to the target results in the emitters being separated from each other by from 0.9 to 1.25 times the Förster radius.

84. The method of claim 74 wherein hybridization of the probe to the target results in the emitters being separated from each other by a distance of within one Angstrom of the Förster radius.

85. The method of claims 74-84 wherein said method is carried out in vivo.

86. The method of claims 85 wherein said target polynucleotide is located inside a cell and said oligonucleotide probes have been introduced into said cell.

87. The method of claim 86 wherein said probes have been introduced into said cell by electroporation.

88. The method of claims 74-84 wherein said probes are attached to a solid support.

89. A method for detecting a disease condition in a patient comprising administering to said patient an effective amount of a probe of claims 14, 34 or 48 and wherein said first and second targeting portions are complementary to nucleotide sequences found in polynucleotides present in said patient and available for contact with said probe as a result of said disease condition.

90. The method of claim 89 wherein said disease is cancer and said target polynucleotide is derived from cell debris produced as a result of said cancer.

91. The method of claim 90 wherein said cancer is a gastrointestinal cancer.

92. The method of claim 89 wherein said disease is caused by an infectious agent and said target polynucleotide is derived from the genome of said infectious agent.

93. The method of claim 92 wherein said infectious agent is a member selected from the group consisting of viruses, bacteria, fungi and protozoans.

94. A method for determining the presence of a target polynucleotide comprising contacting said target polynucleotide with an oligonucleotide probe wherein said probe comprises a first targeting portion and a second targeting portion, wherein said first targeting portion comprises a quencher and an emitter spatially arranged so as to achieve quenching of said emitter and wherein said second targeting portion comprises a member selected from the group consisting of a quencher, a quencher and an emitter, and a harvester and wherein when said member is a quencher and an emitter these are spatially arranged so as to quench said emitter, and wherein said target and said probe are contacted under conditions promoting hybridization of said probe to said target and wherein said hybridization induces at least two conformational changes in said probe oligonucleotide resulting in separation of said quencher, or quenchers, from said emitter, or emitters, so as to produce a detectable signal indicative of hybridization to said target thereby determining the presence of said target polynucleotide.

95. The method of claim 94 wherein said probe further comprises a linker between said first targeting portion and said second targeting portion.

96. The method of claim 94 wherein said probe comprises both an emitter and a harvester and wherein hybridization of said probe to said target results in the emitter and the harvester being separately by a distance between 0.9 and 1.1 times their Förster radius.

97. The method of claim 94 wherein said probe is the probe of claim 13, 26 or 34.

98. The method of claim 94 wherein said probe is attached to a solid support.

99. The method of claim 94 wherein said method is carried out in vivo.

100. The method of claims 99 wherein said method is carried out inside a living cell.

101. The method of claim 99 wherein said method is carried out in an animal.

102. The method of claim 101 wherein said animal is a human patient.

103. The method of claim 101 wherein said probes are administered orally to said animal.

104. The method of claim 103 wherein said probes are part of a pill that comprises a fluorescent detector moiety.

105. An oligonucleotide probe comprising first and second targeting portions and a linker portion, wherein said first targeting portion comprises a quencher and an emitter in sufficiently close spatial proximity to afford quenching of said emitter by said quencher, wherein said second targeting portion comprises a harvester and wherein said harvester and said emitter are separated by said linker portion which linker portion is different from said first or second targeting portion and wherein said harvester is such that it emits light when it is within a selected distance of said emitter.

106. The oligonucleotide probe of claim 105 wherein said first targeting portion comprises at least two internally complementary segments.

107. The oligonucleotide probe of claim 106 wherein said first targeting portion is a hairpin oligonucleotide.

108. The oligonucleotide probe of claim 106 wherein said first targeting portion does not hybridize to said second targeting portion.

109. The oligonucleotide probe of claim 105 wherein said linker portion contains a sequence of nucleotides

110. The oligonucleotide probe of claim 105 wherein said linker portion does not hybridize to either said first or said second targeting portion.

111. The oligonucleotide probe of claim 105 wherein said linker portion is other than an oligonucleotide.

112. The oligonucleotide probe of claim 111 wherein said linker portion is a polymer.
